(19) United States
(12) Patent Application Publication
(10) Pub. No.: US 2003/0050662 A1
(43) Pub. Date: Mar. 13, 2003

(54) Devices for observing and treating body passages

(76) Inventor: T. Anthony Don Michael, Bakersfield, CA (US)

Correspondence Address: BROWDY AND NEIMARK, P.L.L.C., 624 NINTH STREET, NW, SUITE 300, WASHINGTON, DC, 20001-5303, US

(21) Appl. No.: 10/200,498

(22) Filed: Jul. 23, 2002

Related U.S. Application Data
(60) Provisional application No. 60/317,469, filed on Sep. 7, 2001.

(51) Int. Cl. A61M 29/00

(52) U.S. Cl. 606/200

(57) ABSTRACT

A device for treating conditions causing obstructions in a body passage. The device is composed of: a first catheter dimensioned to be insertable into the body passage and having a lateral wall, a proximal end and a distal end; and a first balloon carried by the first catheter and extending outwardly from the lateral wall. The first catheter is provided internally with not more than three fluid conducting passages, including: a blood bypass flow passage extending at least from a first point located between the first balloon and the proximal end to a second point at the distal end and communicating at the first point with a region surrounding the first catheter; a balloon inflation passage communicating with the first balloon; and a delivery/aspiration passage opening at the lateral wall at a location between the first point and the first balloon.

This application claims the benefit of U.S. Provisional Application Ser. No. 60/317,469, filed Sep. 7, 2001.

### BACKGROUND OF THE INVENTION

The present invention relates to devices for treating body passages, and particularly catheter devices. Devices according to the invention are particularly useful for treating obstructions in blood vessels.

A wide variety of catheter devices for performing such operations are already known in the art. In order to be safely introduced into blood vessels, such devices must have relatively small diameters. However, they must also contain a number of lumens to provide at least one blood bypass flow passage, balloon inflation passages and treatment agent delivery passage, each of which must be dimensioned to allow an adequate flow of fluid. Given the constraints on the outer diameter of such a device, provision of the necessary number of passages, or lumens, creates certain difficulties.

Recent data suggests that heart attacks are caused by rupture of atherosclerotic plaques resulting in the formation of occluding clot. Statistically in 86% of heart attacks the obstruction by plaque is less than 70% and in 68% of cases less than 40%. A method to disintegrate clot while reducing or preventing microembolism of the distal circulation could be expected to provide a rapid and effectively treatment of heart attacks. Given the limitation of blood clot dissolving agents, such a device could prove to be life saving.

### BRIEF SUMMARY OF INVENTION

The present invention provides novel treatment devices having a reduced number of lumens, and thus alleviating a number of the problems that exist in the prior art.

A device according to the invention is provided for treating conditions causing obstructions in a body passage and is composed of: a first catheter dimensioned to be insertable into the body passage and having a lateral wall, a proximal end and a distal end; and a first balloon carried by the first catheter and extending outwardly from the lateral wall. The first catheter is provided internally with not more than three fluid conducting passages, including: a blood bypass flow passage extending at least from a first location between the first balloon and the proximal end to a second location at the distal end and communicating at the first location with a region surrounding the first catheter; a balloon inflation passage communicating with the first balloon; and a delivery/aspiration passage opening at the lateral wall at a location between the first location and the first balloon.

The invention further provides an apparatus for treating conditions causing obstruction in a body passage, comprising:

a guiding catheter insertable into the body passage to define a guide passage;

a filter that is movable between a radially contracted condition and a radially expanded condition, the filter being dimensioned to obturate the body passage to block embolic debris when the filter is in the radially expanded condition;

a first sheath having a longitudinal lumen into which the filter can be withdrawn when in the radially contracted condition, the first sheath being dimensioned to be moved along the guide passage; and

an obstruction disintegrating device movable along the guide passage into a position for disintegrating an obstruction in the body passage.

### BRIEF DESCRIPTION OF DRAWING

FIG. 1 is a simplified pictorial representation of one embodiment of a device according to the invention.

FIGS. 2 and 3 are cross-sectional views illustrating the distal ends of two embodiments of devices according to the invention.

FIGS. 4 and 5 are partly pictorial, partly cross-sectional views showing components of a further treatment arrangement according to the invention.

### DETAILED DESCRIPTION OF THE INVENTION

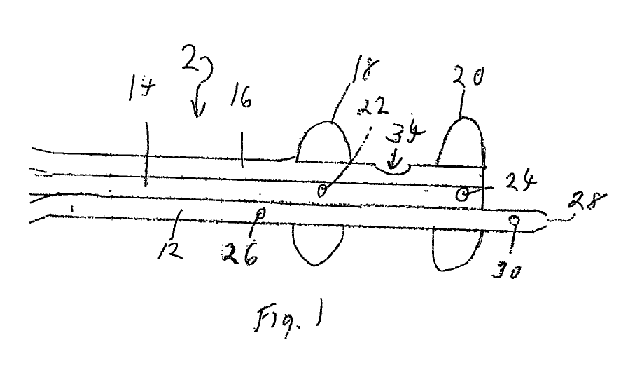

FIG. 1 is a simplified pictorial view illustrating the basic components of a balloon catheter device according to the invention. FIG. 1 is intended to provide an illustration of the basic components of the device and their relation to one another, but does not purport to be a dimensionally faithful illustration of a practical embodiment of the invention.

As shown in FIG. 1, the device according to the invention includes a catheter 2 containing only three lumens, including a blood bypass flow lumen 12, a balloon inflation lumen 14 and a fluid delivery/aspiration lumen 16. The device is further provided with two balloons 18 and 20, each mounted on the outer wall of catheter 2 and each communicating with lumen 14 via a respective fluid passage 22, 24.

Lumen 12 is provided with at least one blood inlet passage 26 located proximally of balloons 18 and 20. The distal end of lumen 12 is provided with at least one axial blood outlet opening 28, and possibly with one or more lateral blood outlet openings 30. Lumen 12 further constitutes a passage for a guidewire (not shown in FIG. 1) that serves to guide catheter 2 within the associated blood vessel.

Lumen 16 is provided with at least one passage 34 via which lumen 16 communicates with a region that surrounds catheter 2 and that is enclosed between balloons 18 and 20.

Both lumens 14 and 16 are closed at their distal ends.

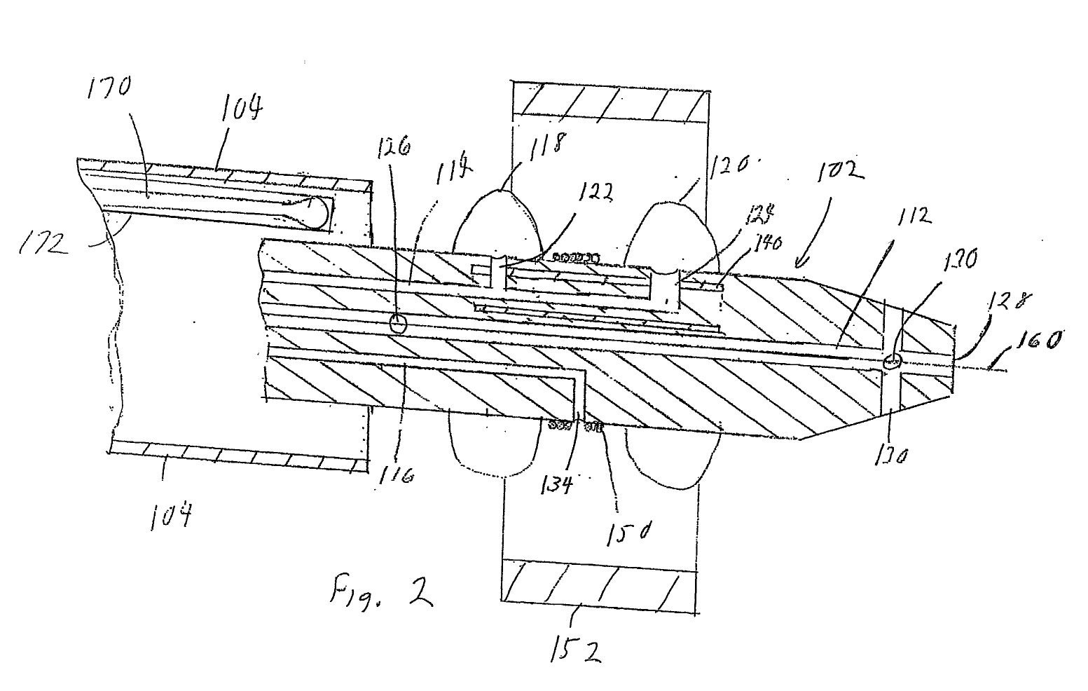

FIG. 2 is cross-sectional view of one practical embodiment of a catheter according to the invention, which corresponds functionally to the device illustrated in FIG. 1. Elements corresponding to those shown in FIG. 1 are provided with corresponding reference numerals proceeded by a “1”. Thus, FIG. 2 shows a catheter 102 that may be introduced into a blood vessel through a guiding catheter 104, or a hypotube, as is conventional in this art.

Catheter 102 includes a blood bypass flow lumen 112, a balloon inflation lumen 114 and a delivery/aspiration lumen 116. Catheter 102 carries, on its outer surface, two balloons 118 and 120.

Lumen 114 communicates with balloons 118 and 120 via respective passages 122 and 124. In order to equalize the pressure of the fluid supply to each of the balloons, passage 124 may have a larger cross section then passage 122. In addition, balloon 120 may be fabricated to have a higher compliance, i.e. a greater elasticity, than balloon 118. However, when the inflation fluid is a liquid, the fluid pressure in passages 122 and 124 will be substantially equal. Balloon 118 may be a low compliance pressure balloon. Alternatively, by making balloons 118 and 120 identical, i.e., of the same material and with the same dimensions, and locating each passage 122 and 124 symmetrically with respect to its associated balloon, i.e., so that the center line of the passage is equidistant from the proximal and distal lines of attachment of the balloon to the catheter, virtually equal inflation of the balloons can be realized.

Lumen 112 communicates with the region surrounding catheter 102 at a location proximal to balloon 118, via one or more inlet passages 126. The distal end of lumen 112 communicates with the region outside catheter 102 via an axial outlet opening 128 provided at the distal end of catheter 102 and possibly via one or more lateral outlet openings 130 that open at the lateral wall of catheter 102.

Lumen 116 communicates with a region surrounding catheter 102 and located between balloons 118 and 120 via a flow passage 134.

To assure that lumen 114 remains open in the region between balloons 118 and 120, catheter 102 may be strengthened in that region either by making catheter 102 slightly thicker in that region or by embedding a metal part, such as a titanium tube 140, at the time catheter 102 is formed by extrusion, or the catheter may be fabricated from a suitable plastic impregnated with titanium.

The device may be completed by a copper MRI transmit-receive coil 150 wound around the outer surface of catheter 102 in the region between balloons 118 and 120. Leads (not shown) for coil 150 may extend along catheter 102 to the proximal end thereof (not shown) for connection to conventional MRI components. This will allow MRI apparatus to be used to image the portion of the blood vessel wall between balloons 118 and 120 in order to provide information allowing proper positioning of catheter 102 and about the condition of the blood vessel wall in the region to be treated. For this purpose, the patient would be positioned so that the part of the patient's body that contains the vessel to be treated is enclosed by an annular magnet 152 that is needed to effect imaging and that is a standard component of existing MRI apparatus.

The device is completed by a guidewire 160 that extends through lumen 112. All of the illustrated lumens extend to a manifold (not shown) at the proximal end of catheter 102 in accordance with conventional practice in this art.

Catheter 102 can be introduced into a blood vessel to be treated in a conventional manner by first inserting guidewire 160 through guiding catheter 104 and then introducing catheter 102 over guidewire 160, i.e., by placing lumen 112 around guidewire 160 and through guiding catheter 104. Catheter 102 is advanced over guidewire 160 until reaching the location of the blood vessel where a treatment is to be performed. The positioning of catheter 102 may be aided by images produced by MRI equipment, as described above, and/or by providing radiopaque markers on either side of each balloon and employing fluoroscopic guidance.

When catheter 102 is properly positioned, balloons 118 and 120 are inflated by introducing inflation fluid through lumen 114 and passages 122 and 124. A variety of treatments may then be performed, such as disclosed in my issued U.S. Pat. No. 5,460,601, the contents of which are incorporated herein by reference. In the region between balloons 118 and 120, treatments with genes and chemotherapeutic drugs, thrombolytic drugs, anticoagulants and other forms of drug therapy, including treatments to passivate a clot site, can be carried out. Since the region of the blood vessel between balloons 118 and 120 is isolated from the remainder of the blood flow system, small quantities of a treatment agent can provide a high concentration at the treatment site.

The use of a single lumen for inflation of balloons 118 and 120 simplifies the structure of catheter 102 and provides additional space for the other lumens. It will be understood that the drawings do not necessarily show the lumens to scale and that the lumens can be given relatively large cross sections.

Since catheter 102 can be made smaller than prior art catheters having given lumen cross-sectional dimensions, it would further be possible to introduce a probe 170 that is housed within a sheath 172 of an ultrasonic system between catheters 102 and 104 and to bring the tip of this probe to the region to be treated, by extending the probe out of sheath 172 in order to supply ultrasonic energy that can potentiate the dispersal of drugs and genes into, and even through, the vessel wall at the treatment site. The ultrasonic energy may also be used to disintegrate plaque or clot. If confronted with certain conditions, such as an acute heart attack caused by extruded thrombus or clot, a suitable dissolution “cocktail” could also be introduced into the treatment region via lumen 116 and flow passage 134. One ultrasonic system that would be suitable for this purpose is disclosed in U.S. Pat. No. 4,870,953, the contents of which are incorporated herein by reference. In such a system, sheath 172 is provided with an annular passage for the flow of cooling medium to prevent overheating of probe 170 while in operation.

The tip of probe 170 can be moved into the region between balloons 118 and 120 either before the balloons have been inflated, or, if the balloons have already been inflated, they can be deflated briefly to allow the tip of probe 170 to be positioned between them. After the tip of probe 170 has been positioned, balloons 118 and 120 can be inflated. Balloon 118 will form a reasonably effective seal despite the presence of probe 170.

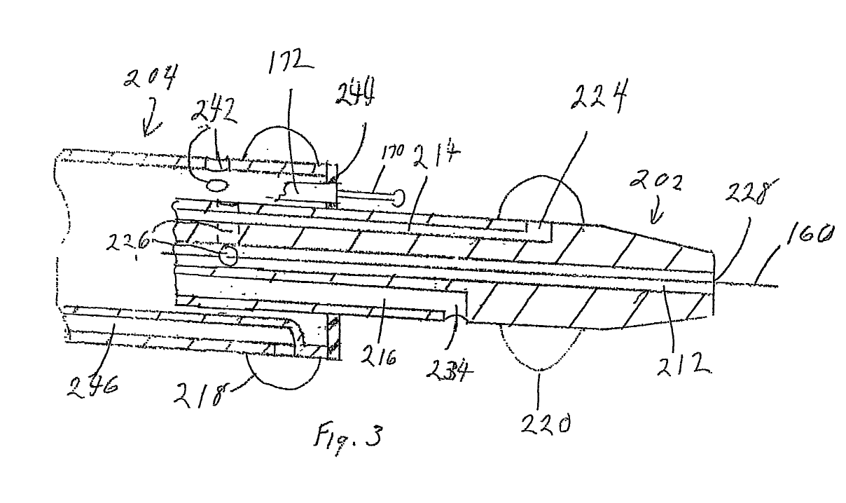

FIG. 3 is a cross-sectional view of a second practical embodiment of the invention composed of a catheter 202 and an outer guiding catheter 204. Catheter 202 is provided with three lumens: a blood bypass flow lumen 212, a balloon inflation lumen 214 and a delivery/aspiration lumen 216.

Catheter 204 carries a first balloon 218 and catheter 202 carries a second balloon 220, these balloons being identical to balloons 118 and 120 of the embodiment shown in FIG. 2.

An inflation fluid flow passage 224 extends laterally between lumen 214 and the interior of balloon 220. Lumen 212 communicates with the region surrounding catheter 202 via one or several blood inlet flow passages 226 located proximally of balloon 220. The distal end of lumen 212 has at least an axial blood outlet opening 228 and may have one or more lateral blood outlet openings, as in the embodiment of FIG. 2. Lumen 216 communicates, via a passage 234, with the region surrounding catheter 202, at a location between balloon 220 and passages 226.

Catheter 204 is provided with one or several blood inlet flow openings 242 and carries, at its distal end, an annular seal member 244 that bears against the outer surface of catheter 202 to seal the annular space between catheters 202 and 204. Catheter 204 also carries a thin tube, or lumen, 246 for supplying inflation fluid to balloon 218.

Catheter 202 is displaceable in the axial direction relative to catheter 204 in order to vary the spacing between balloons 218 and 220, to thereby vary the size of the isolated treatment region between those balloons. When the arrangement shown in FIG. 3 is introduced into a blood vessel and balloons 218 and 220 have been inflated to isolate the region between those balloons, a flow of blood will be maintained over the path defined by openings 242, passages 226, lumen 212 and blood exit opening 228. Balloons 218 and 220 may have the characteristics described above with respect to balloons 18 and 20 of FIG. 1 and balloons 118 and 120 of FIG. 2.

The device shown in FIG. 3 may be utilized in the same manner as the similar device disclosed in my issued U.S. Pat. No. 5,342,306, the entire contents of which are incorporated herein by reference. An essential difference between the device disclosed in that patent and that of the present invention resides in a reduction in the number of passages, or lumens, in catheter 202. Specifically, in contrast to the arrangement disclosed in the above-cited issued patent, catheter 202 according to the present invention is provided with only three lumens, thereby reducing the complexity of the catheter and allowing the provision of larger lumen cross-sections for a given catheter diameter.

Catheter 202 may also be provided with a MRI coil comparable to coil 150 of the embodiment shown in FIG. 2, along with other MRI components.

All of the above-described embodiments of the invention are used in a similar manner in that they may all be introduced into the blood vessel via a guiding catheter, which may be catheter 104 of FIG. 2, or an additional guiding catheter with respect to the embodiment of FIG. 3. After being brought to the desired location in the blood vessel, the two balloons are inflated to create an isolated treatment region, and a suitable treatment drug may be introduced via lumen 16, 116, or 226. During the course of the treatment, fluid may be periodically withdrawn via that lumen for analysis purposes. More specifically, devices according to the invention can be utilized to perform treatments as described in my issued U.S. Pat. No. 5,306,249, the entire contents of which are incorporated herein by reference.

If it is desired to perform a treatment with ultrasonic energy, the distal end of sheath 172 can be fitted in an opening provided in seal member 244, the opening being dimensioned to form a seal with the outer surface of the sheath.

Devices according to the invention can be used in conjunction with a variety of energy sources for disintegrating blockages including ultrasound devices, as mentioned above, laser devices and mechanical devices.

The embodiment shown in FIG. 3 can be used effectively in the treatment and removal of an elongated clot that may have formed in a blood vessel. Such clots are difficult to remove because of their relatively long length, possibly as long as 3 cm. With the device of FIG. 3, catheter 202 can be extended out of catheter until balloons 218 and 220 straddle the clot. Then balloon 220 may be inflated and catheter 202 may be retracted partially into catheter 204, while catheter 204 remains stationary, to drag balloon 220 along the vessel wall and push the clot toward balloon 218. This operation is preferably performed using a high compliance, or soft, balloon as balloon 220. After the clot has thus been longitudinally compressed, it may be broken up more efficiently by ultrasonic, laser, or mechanical action, possibly in combination with chemical treatment, and the clot material can be withdrawn via lumen 216. The procedure described above can be performed using a known single balloon catheter in place of catheter 202.

The effectiveness of balloon 220 in pushing the clot toward balloon 218 can be enhanced by constructing balloon 220 so that when inflated it presents a concave surface toward balloon 218. Such a form of construction is shown in FIG. 8 of my issued U.S. Pat. No. 5,195,955, issued on Mar. 23, 1993, the disclosure of which is incorporated herein by reference. However, whereas the balloon shown in the patent inflates eccentrically relative to the axis of the catheter on which it is mounted, balloon 220 will be constructed to inflate concentrically.

For treatment of an acute heart attack where an arterial blockage is present, a catheter of the type disclosed herein could be inserted as a first treatment step and pushed across the blockage to provide immediate, temporary restoration of at least a limited blood flow.

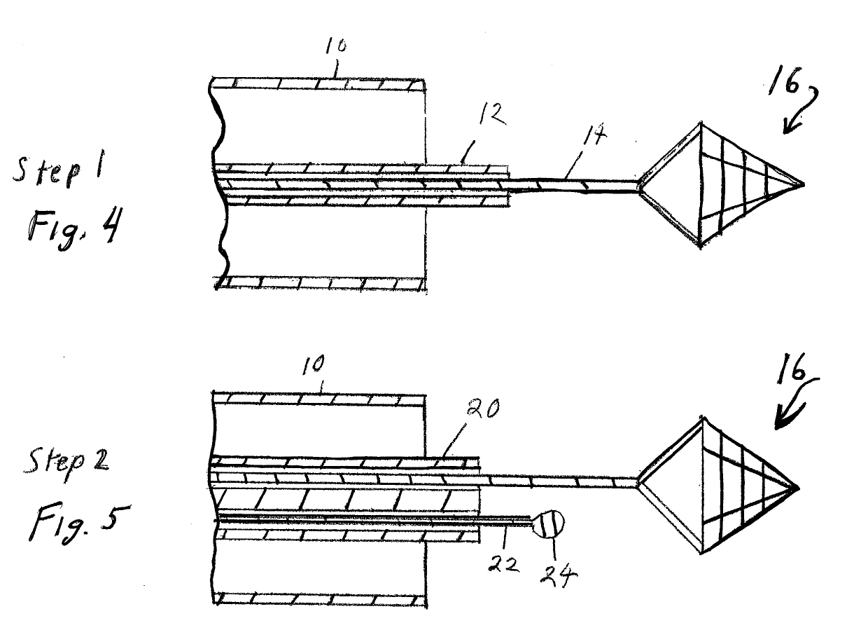

A further embodiment of the invention will be described with reference to FIGS. 4 and 5. The arrangement shown in FIG. 4 is composed of a guiding catheter 410 enclosing a sheath, or catheter, 412. Sheath 412 is formed to have a longitudinally extending lumen that extends fully from the proximal end (not shown) to the distal end thereof. This lumen is provided to contain a filter support wire 414 and a filter 416 carried at the distal end of wire 414.

The arrangement shown in FIG. 5 includes a sheath, or catheter, 420 having two longitudinal lumens, each of which extends completely from the proximal end (not shown) to the distal end thereof. One lumen in catheter 420 is provided to receive the same support wire 414 and filter 416 as the lumen in sheath 412, as will be explained in detail below. The other lumen of catheter 420 is provided to guide a wire 422 that is provided at its distal end with a bulbous tip 424. Tip 424 constitutes the output end of an ultrasonic energy generator having a source of ultrasonic vibration (not shown) connected to the proximal end of wire 422.

In an exemplary embodiment of the present invention, guiding catheter 410 may have a 6 Fr internal diameter, sheath 412 may have a one millimeter outer diameter, catheter 420 may have a 1-2 mm outer diameter and wire 414 may have a 0.014 inch outer diameter. The dimensions of wire 422 and its tip 424 will be selected in the basis of principles governing the design of ultrasonic vibration sources.

Wire 422 and tip 424, as well as the ultrasonic vibration generating components, may be constructed as disclosed in issued U.S. Pat. No. 4,870,953, the disclosure of which is incorporated herein by reference.

Filter 416 is composed of a flexible metal framework, or armature, carrying, at its distal side, which is at the right-hand side in FIGS. 4 and 5, a sheet of porous filter material having a pore size that allows passage of blood while blocking the passage of debris resulting from the disintegration of clots or plaque. Preferably, the armature is made of a memory metal, such as Nitinol® and may be constructed in the manner disclosed in co-pending application Ser. No. 09/803,641, filed Mar. 12, 2001, the disclosure of which is incorporated herein by reference, and particularly the form illustrated in FIG. 7A thereof.

In order to treat an obstruction in a blood vessel, firstly, guide catheter 410 is introduced into the vessel upstream of the obstruction. Sheath 412 may be introduced simultaneously with, or subsequent to the introduction of, catheter 410. At the time that sheath 412 is introduced, filter 416 may be retracted into the distal end of the lumen in that sheath. After sheath 412 has been positioned, filter 416 is advanced out of the lumen in sheath 412 by moving wire 414 in the distal direction. Filter 416 is constructed to expand when not subjected to a compression force, i.e. to be unstressed when in its expanded state.

Normally, it will be desired to position filter 416 downstream of the obstruction before it is expanded. This is achieved by advancing sheath 412 to the right, past the obstruction, before advancing filter 416 out of and away from sheath 412. In FIG. 4, filter 416 is shown in its partly expanded state and it should be appreciated that filter 416 is not necessarily illustrated to scale.

After filter 416 has been deployed, or expanded, to extend across the blood vessel downstream of the obstruction, wire 422 (shown in FIG. 5), which normally has an outer diameter of 0.014 inch, can be introduced into guide catheter in a variety of ways in order to bring tip 424 into position for disintegrating the obstruction. According to one possibility, while sheath 412 is withdrawn, a separate guide wire (not shown), usually also with an outer diameter of 0.014 inch, can be introduced through guide catheter 410 so as to extend beyond the obstruction and tip 424 can be provided with a through bore through which the guide wire passes to act as a guide for the tip. Then wire 422 can be advanced to bring tip 424 to the treatment site while being guided by the guide wire. An ultrasonic or other clot disintegrating device having a tip provided with such a through bore is disclosed in my copending U.S. Provisional Application Ser. No. 60/317,472, filed on Sep. 7, 2001. According to a second possibility, sheath 412 can be withdrawn, the proximal end (not shown) of wire 414 can be threaded through the through bore in tip 424 and wire 422 can be advanced along the guide passage formed by guide catheter 410 while wire 414 acts as a guide wire for tip 424. According to a third possibility, with sheath 412 withdrawn, wire 422 surrounded by a sheath of its own may be advanced through the guide passage defined by guide catheter 410 without use of a guide wire in order to bring tip 424 to a position in which it will perform a disintegration operation on the obstruction, as described in the above-cited issued U.S. Pat. No. 4,870,953. After tip 424 has been brought into position according to any one of these possibilities, ultrasonic vibrations are imparted to tip 424 to disintegrate the obstruction, which is usually constituted by clot. As the obstruction is disintegrated, the resulting debris will be collected in filter 416, being conveyed toward the distal end thereof by blood flowing in the blood vessel. After the disintegration operation has been completed, sheath 412 will be advanced distally while wire 414 is held stationary. This will cause filter 416 to be contracted into the lumen in sheath 412. This contraction is aided by a camming action preformed by the distal end of sheath 412 on the proximal struts of filter 416. Debris will thus be trapped within the filter and within sheath 412. Then all components may be withdrawn to complete the treatment.

Alternatively, as shown in FIG. 5, after filter 416 has been deployed, sheath 412 may be withdrawn and replaced by sheath 420 having one lumen that is fitted around wire 414 while sheath 412 is being introduced through guide catheter 410 and a second lumen containing wire 422. During introduction of sheath 420, one lumen of sheath 420 will be advanced around wire 414, while wire 422 will be disposed in the other lumen thereof.

Here again, after disintegration of an obstruction has been completed, catheter 420 will be advanced in the distal direction in order to cause catheter 420 to slide around filter 416, thereby collapsing the filter into the associated lumen in catheter 420. After filter 416 has been brought completely into that lumen, catheter 410 and sheath 420 are withdrawn from the blood vessel and treatment is completed.

In further accordance with the invention, a treatment process can be carried out using, sequentially, either one of the devices shown in FIGS. 2 and 3 and the device shown in FIGS. 4 and 5. A clot removal treatment can be composed of three steps, passivation of the clot site, as described earlier herein, clot disintegration and debris removal. All three steps can be performed with either of the devices shown in FIGS. 2 and 3, while clot disintegration and debris removal can also be performed with the device shown in FIGS. 4 and 5. In certain situations, e.g., if a life-threatening situation requiring immediate clot removal is presented, the device shown in FIGS. 4 and 5 would be used first to effect clot disintegration and debris removal. Then, that device could be withdrawn and the device of FIG. 2 or 3 introduced to perform passivation, which serves to cool the treatment site. Otherwise, the device of FIG. 2 or 3 would be introduced first to perform passivation, after which that device would be withdrawn and the device shown in FIGS. 4 and 5 would be introduced to effect clot disintegration and debris removal. In either of these procedures, it would not be necessary to introduce probe 170 with the device of FIG. 2 or 3. Particularly when the device of FIG. 2 is used in one of these procedures, it would not be necessary to withdraw guide catheter 104 or 410 of the first device that is used and that guide catheter could be used with the other device.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

What is claimed is:

1. A device for treating conditions causing obstructions in a body passage, comprising:
a first catheter dimensioned to be insertable into the body passage and having a lateral wall, a proximal end and a distal end, said first catheter being insertable into the body passage via said distal end; and
a first balloon carried by said first catheter and extending outwardly from said lateral wall,
wherein said first catheter is provided internally with not more than three fluid conducting passages, including: a blood bypass flow passage extending at least from a first location between said first balloon and said proximal end to a second location at said distal end and communicating at the first location with a region surrounding said first catheter; a balloon inflation passage communicating with said first balloon; and a delivery/aspiration passage opening at said lateral wall at a location between said first location and said first balloon.

2. The device of claim 1 further comprising a second balloon carried by said first catheter and extending outwardly from said lateral wall, said second balloon being located between said first location and said first balloon and communicating with said balloon inflation passage.

3. The device of claim 2 wherein said first balloon has a compliance higher than that of said second balloon.

4. The device of claim 2 wherein said first catheter comprises rigidifying means for preventing compression of said balloon inflation passage between said first and second balloons.

5. The device of claim 1 further comprising a MRI transmit-receive coil carried by said lateral wall.

6. The device of claim 2 wherein said blood bypass flow passage further extends from said first location to said proximal end of said first catheter, and further comprising a guide wire extending through said blood bypass flow passage.

7. The device of claim 1 wherein said blood bypass flow passage further extends from said first location to said proximal end of said first catheter, and further comprising a guide wire extending through said blood bypass flow passage.

8. The device of claim 1 further comprising:
a second catheter having a lateral wall surrounding said first catheter and movable relative to said first catheter in a direction between said proximal and distal ends of said first catheter, said second catheter being provided with a second balloon inflation passage; and
a second balloon carried by said second catheter and communicating with said second balloon inflation passage.

9. The device of claim 8 further comprising a sealing element forming a fluid seal between said second catheter and said lateral wall of said first catheter.

10. The device of claim 9 wherein said first location of said blood bypass flow passage is enclosed by said second catheter, and said second catheter is provided with blood flow passages extending through said lateral wall of said second catheter at a location that is proximal of said sealing element for permitting blood flow from a region surrounding said second catheter to said first location of said blood bypass flow passage.

11. The device of claim 8 wherein said blood bypass flow passage further extends from said first location to said proximal end of said first catheter, and further comprising a guide wire extending through said blood bypass flow passage.

12. The device of claim 1 in combination with a probe of an ultrasonic energy generating system, said probe having an output tip that is positionable adjacent said catheter between said first location and said first balloon.

13. The device of claim 1, further comprising an MRI coil wound around said first catheter and connectable to MRI apparatus for permitting observation of the body passage.

14. An apparatus treating conditions causing obstruction in a body passage, comprising:
a guide catheter insertable into the body passage to define a guide passage;
a filter that is movable between a radially contracted condition and a radially expanded condition, said filter being dimensioned to obturate the body passage to block embolic debris when said filter is in the radially expanded condition;
a first sheath having a longitudinal lumen into which said filter is withdrawn when in the radially contracted condition, said first sheath being dimensioned to be moved along said guide passage; and
an obstruction disintegrating device movable along said guide passage into a position for disintegrating an obstruction in the body passage.

15. The apparatus of claim 14 wherein said first sheath has a second longitudinal lumen for guiding said obstruction disintegrating device.

16. The apparatus of claim 14 wherein said first sheath has only said longitudinal lumen, and further comprising a second sheath useable in place of said first sheath and having a first longitudinal lumen into which said filter may be withdrawn and a second longitudinal lumen for guiding said obstruction disintegrating device.

17. The apparatus of claim 14 further comprising a filter support wire connected to said filter and arranged to extend through said longitudinal lumen of said first sheath.

18. The apparatus of claim 14 wherein said filter is made of a flexible, radially compressible, radially expandable material, said filter being formed to have a conical configuration when radially expanded and being composed of a plurality of circumferential, curved strips and a plurality of longitudinal struts interconnecting said strips, and a sheet of filter material secured to said strips and struts.

19. The apparatus of claim 14 wherein said obstruction disintegrating device is an ultrasonic acoustic wave generator.
